(19) United States
(12) Patent Application Publication
(10) Pub. No.: US 2021/0104327 A1
(43) Pub. Date: Apr. 8, 2021

(54) Risk Assessment from Modulated Sequences by Deconvolution of Reference Specimen Profiles

(71) Applicant: Aaron Zhang-Chen, San Diego, CA (US)

(72) Inventor: Aaron Zhang-Chen, San Diego, CA (US)

(73) Assignee: Genenius Genetics, San Diego, CA (US)

(21) Appl. No.: 17/023,637

(22) Filed: Sep. 17, 2020

Related U.S. Application Data
(60) Provisional application No. 62/911,343, filed on Oct. 6, 2019.

(51) Int. Cl. G16H 50/30; G16B 50/10; G16B 30/00; G16B 35/00; C12Q 1/68

(52) U.S. Cl. CPC G16H 50/30; G16B 50/10; C12Q 1/68; G16B 35/00; G16B 30/00

(57) ABSTRACT

Databases of specimen profiles of reference loci, and methods of querying the databases with samples to detect and assess changes in the physiological state of an organism. Reporting certain changes in a state, as well as providing a risk assessment.

### CROSS-REFERENCE TO RELATED APPLICATIONS

This application claims the benefit of priority of U.S. provisional application Ser. 62/911,343, filed Oct. 6, 2019, the contents of which are incorporated herein in its entirety.

### TECHNICAL FIELD

Analyzing biological samples for bioinformatic comparison with reference databases.

### SUMMARY OF THE INVENTION

The present invention provides databases of genetic information from different reference specimens from various sample types, and from pathological and nonpathological states. For each type of specimen, the database contains profiles of information from different loci.

The invention also provides methods for querying the databases with samples that are taken from an organism. Multiple samples—such as samples taken over months—can be queried from the same organism. Changes in the physiological state of the organism can be detected by comparison and deconvolution of the query results.

Also provided are methods for assessing changes in the state of organism and reporting such changes, such as by providing an overall risk assessment score.

### BRIEF DESCRIPTION OF THE DRAWINGS

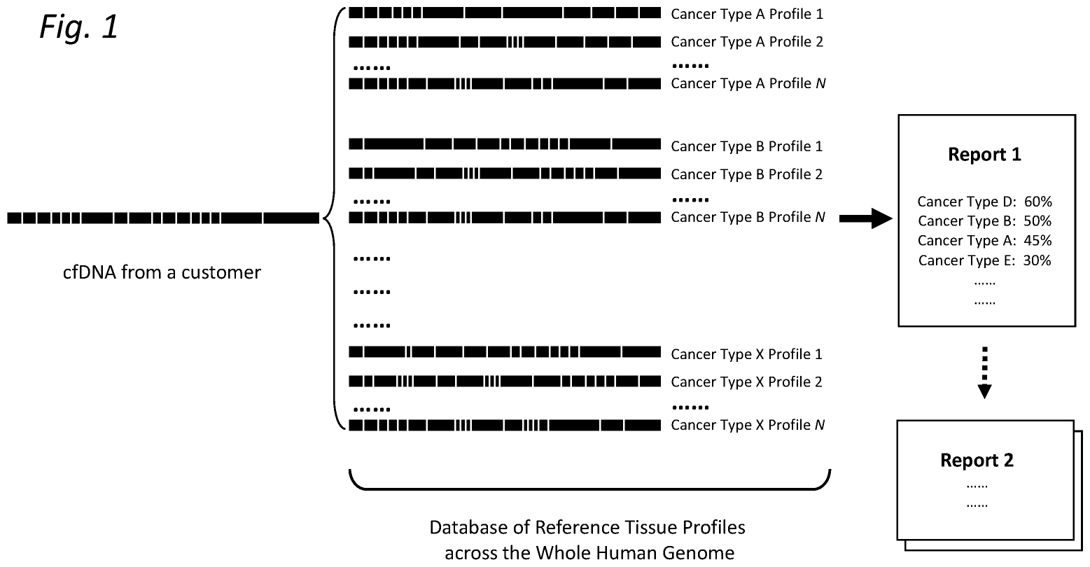

The middle of FIG. 1 depicts a set of whole genome profiles from reference specimens (e.g. “Cancer Type A Profile 1”), which are values of genetic information at various loci (indicated by short white segments against a black background) for a specimen type. For example, a profile for lung cancer can be represented by a set of loci information across the human genome from a lung sample, whether pathological or nonpathological. As shown, a particular “Cancer Type A” can have multiple profiles (1, 2, . . . N). Similarly, a “Cancer Type B” (e.g. ovarian) can be represented by profiles from healthy and nonhealthy ovarian samples. Together, the profiles of specimen loci can be stored in a database that can be queried.

The left of the figure depicts a profile of genetic information from a sample from an organism where the sample shown is a sample of cell-free DNA (cfDNA). The profile can be queried against the database of profiles to generate a report of comparisons with specimen profiles. The information derived from the comparisons with particular specimen types can be summarized in a report shown on the right (Report 1). In some embodiments, scores for similarity or weighted comparison can be reported relative to each cancer or specimen type. Scores can be reported for multiple samples and for multiple time points (Report 2, etc.).

### DETAILED DESCRIPTION OF THE INVENTION

A disease state may be conceptualized as a deviation from an idealized healthy state or from a defined normal physiological range. Previous assessments of the state of an organism suspected of having a disease state used comparisons of the current state of the organism with the signs of a known disease, such as by comparison with the physical indicia of diseased organs, tissues, fluids, or cells. With the plummeting cost of sequencing, the indicia of disease can encompass genetic information from diseased specimens, such as malignant tumors, and individual tumor cells, whose individual genomes can evolve as they compete for resources within the tumor environment. Under this framework, disease is detected by comparing the state of an organism with a reference set of markers for cells and specimens that have been characterized pathologically as being diseased.

The present invention provides profiles of genetic information from reference specimens. The specimens can be tissues, body fluids, or other samples from healthy individuals, or they can be samples from living or dead individuals where the specimens that have not been selected as representative of a disease or pathological condition. A reference specimen may be considered healthy or nonpathological for the purposes of a genetic profile if it harbors a latent or unknown disease or infection, or suffered from physical trauma that does not affect its genetic information. Optionally, profiles from known pathology samples can also be included for reference in the database.

Sets of the profiles can be organized into databases, which can be organized and searched by defined criteria. The information in the databases can be accessible through a database management system. Examples include relational and non-relational database languages and systems, such as SQL.

In the database of the invention, the profiles can be described for convenience as belonging to “Cancer Type A” in the sense that a healthy sample taken from a certain specimen is characteristic of the specimen that is malignant. Thus, the detailed genetic information from a reference sample of healthy lung specimen can be used to build a profile for lung cancers. There are many types of lung cancer, however, and the classification of such cancers continues to evolve. For example, lung cancers can be categorized as small-cell lung carcinoma types and non-small-cell lung carcinoma types (with subtypes adenocarcinoma, squamous-cell carcinoma, and large-cell carcinoma). Other types of lung cancers include carcinoid tumors, bronchial gland carcinomas, and sarcomatoid carcinomas. Many cancers can also be combinations of the various subtypes as presently defined. It is therefore desirable for the database to contain profiles taken from many lung samples to provide a wide informational coverage for different types of lung diseases. In FIG. 1, this is shown as Profiles 1, 2, . . . N, which represent profiles of genetic information from N samples of reference specimen, where the specimen is characteristic of “Cancer Type A”, e.g. lung specimen profiles for various cancers of the lung.

The number of reference specimen types having a profile in the database can be 2, 3, 4, 5, 10, 20, 50, 100, 200, 500, 1000, 2000, or 5000, or more. Similarly, a specimen type can be represented by profiles from 2, 3, 4, 5, 6, 7, 8, 9, 10, 12, 14, 16, 18, 20, 25, 30, 35, 40, 50, 75, 100, 200, 500, or 1000 or more samples, whether healthy or pathological. A database can have 2, 3, 4, 5, 10, 20, 50, 100, 200, 500, 1000, 2000, 5000, 10,000, 20,000, 50,000, or 100,000 or more profiles or reference specimen types.

The reference specimens can be fresh samples and can be from specimens that are intact or have been treated, such as a tissue or cell lysate. Other reference samples can be collected from autopsies, preserved samples, or formalin-fixed paraffin-embedded (FFPE) samples, any of which are optionally characterized pathologically. The DNA from reference specimens can be obtained and prepared for sequencing by commercially available methods, including treatment to remove non-nucleic-acid contaminants.

The nucleic acids include DNA, such as nuclear or mitochondrial DNA. The RNA of specimen can also be collected for analysis, such as mRNA, rRNA, tRNA, siRNAs, antisense RNAs, circular RNAs, or long noncoding RNAs, circular RNA, or modified RNA, or non-nucleic-acid components that are expressed in the specimen. In another embodiment, the genome of the cells in a reference specimen is analyzed in parallel with expression analysis and screening against a panel of antibodies to build a more completely characterized profile. Some treatments include isolation of the nuclei from cells or purification of fractions of chromosomal or mitochondrial DNA through enzymatic treatment, such as with one or more nucleases, proteases, or lipases, or their inhibitors. Such treatments can be controlled for time, temperature, ionic strength, steric effects, and pH to achieve the desired purification under comparable conditions. Other treatments include removal of cellular components such as cytoplasm and mitochondria, unless information from the mitochondria is specifically desired for the profile. Individual treatments can be partial, complete, or combined with other treatments.

The profiles contain genetic and other information from the reference specimens or cells, such as the values at various loci in their genomes. The information can be genetic (i.e. the naturally occurring nucleotide at a locus), but can also include epigenetic information, such methylation and other chemical modifications that were found at a locus in the reference specimen. Modifications in methylation, for example, can be detected by dividing a sample into one aliquot for processing with bisulfite conversion (to convert cytosine to uracil, while leaving 5-methylcytosine intact) and another aliquot for processing without conversion, so that the results from the two aliquots can be compared to indicate the presence of 5-methylcytosine.

The number of loci in an individual profile can be 100, 200, 500, 1000, 2000, 5000, 10,000, 20,000, 50,000, 100,000, 200,000, 500,000 or more. The information can be obtained by sequencing the whole genome of the sample de novo, or by targeted sequencing of regions of interest, such as biomarkers having known associations with cancer or other diseases or conditions. The number and particular loci information of a profile may differ from reference sample to sample and from specimen type to type.

The information at a locus may be for a single nucleotide (such as a SNP) or for a sequence (such as a dinucleotide or longer variable sequence or the presence of a repeated sequences at a locus). Moreover, the information for a locus in a database may also include the abundance (or inclusion within a range of values or other statistical properties) of a particular SNP or sequence among a set of profiles.

Having provided a database of profiles from reference specimen samples, the invention also provides methods for obtaining a sample from an organism or individual. The sample can be hair, skin, or from saliva or a buccal swab for epithelial cells. More particularly, the sample can be from a specimen of interest related to potential disease states, such as lung, blood, breast, head and neck, gastrointestinal tissues, kidney, prostate, liver, and cervix. The sample can be from an observed or suspected tumor, or multiple samples can be taken from different parts of the tumor. Other examples include whole or fractionated blood samples, plasma, serum, lymph, and cerebrospinal fluid. The body fluids can also be processed to enrich for or obtain cell-free DNA (cfDNA), which can include subpopulations of circulating nucleic acids shed from tumor (ctDNA), mitochondria (ccf mtDNA), or fetal or placental cells (cffDNA). The length of nucleic acids analyzed from the individual's sample can vary in length depending on the process. Particular lengths can include 20, 40, 60, 80, 100, 120, 140, 160, 180, 200, 220, 240, 260, 280, 300, 320, 340, 360, 380, or 400, 500, 600, 700, 800, or 1000 bases or more, and can be in any range of these lengths. These samples can provide a snapshot of the physiological state of the individual when the sample was taken, providing information that is a phenotypic expression of the individual's genotype. Thus, a profile of an individual's cfDNA may be described as a manifestation of a cell-free DNA phenotype that can be mined and compared for useful clinical, health, and wellness information.

The genetic information from the individual's sample can then be compared with the reference profiles in the database. For example, the sequencing information can be compared with the values at the set of loci in the profiles. This can be performed by aligning the sequences to different regions of the reference genomes. The comparison can identify similarities and differences from the corresponding loci in the profiles, where similarities to a healthy profile can indicate a healthy state or the absence of a disease involving that reference specimen sample. Conversely, differences from the healthy profile can suggest an unhealthy state, where greater numbers of differences can suggest an unhealthy state more strongly. Algorithms can be developed and used to perform the comparisons, such as formulas for assigning weights of significance to individual loci and sets of loci.

The usefulness of the invention is compounded when samples are taken from the individual over time. Changes in an individual's correlations with the reference profiles can be associated with changes in the physiology of the individual. By taking a sample at one or more subsequent time points, the comparisons can be evaluated over course of days, weeks, months, or years to provide a moving picture of the individual's physiological state.

Reports at individual time points can show a status relative to the reference profiles and be reported relative to a predetermined or generic threshold level for an expected population. An individual's values may be relatively high or low compared to the rest of the population and still not reflect a pathological state. Thus, a series of reports over time can establish a personalized baseline level for the individual and subsequent testing can further reveal progressive changes in the individual's health that should prompt attention. Accordingly, the ratio of change that may be reported as significant can vary between about 5%, 10%, 20%, 25%, 33%, 50%, 66%, 75%, 80%, about equal amounts, 120%, 133%, 150%, 175%, 2×, 2.5×, 3×, 4×, 5×, and 10× relative to each other, including ranges of these ratios. Sudden changes may signal a need for immediate attention.

The changes may correlate directly with the type of specimen of the reference profile, for example the development of a duodenal ulcer with a change in correlation to duodenal epithelial cells, or related to the site of the ulcer, such as the muscularis mucosae and lamina propria or other layers.

On the other hand, a rapid change may be associated with the onset of a pathological state which may not necessarily be conventionally related to the specimen. Through whole-genome sequencing of the reference specimens, the invention can be agnostic as to the etiology of the change or the relatedness of the reference specimen. The early development of a tumor in one part of the body may provoke a change in different or remote cells or a change in the number or composition of cfDNA fragments. For example, a change relative to a reference specimen may be indicative of infection, inflammation or damage due to exercise, chemotherapy, or alcohol or other substance abuse. Other changes may be related to physical degeneration of healthy tissue or cumulative changes such as build-up of vascular plaque or atherosclerosis. Yet other conditions that can be detected by changes relative to reference specimen profiles include allergic and other immune responses, particularly autoimmune responses, which can involve changes relative to multiple reference specimen profiles. Reference to multiple profiles or a series of baseline measurements can also be useful when an individual is a female of menstruating age, experiencing menarche, or perimenopausal. In some cases, tracking changes may be associated with events in the menstrual cycle, such as ovulation, or increased or decreased fertility.

Other physiological changes that can involve multiple reference specimens include cancers, as discussed above, such as early and late stage cancers. The cancers can be grouped conventionally by stages for primary tumor size (when solid), involvement of nearby lymph nodes, and extent of metastasis. Because the detected change in state need be conventionally correlated with the tissue of the reference profile, the invention is agnostic as to mechanism, and can reveal indirect or subtle associations, such as those found in non-Western medical systems. Traditional Chinese Medicine, for example, associates disease states with alterations in vital energy flows, which can “circulate” through traditional meridians that are associated with organs and body systems. Without relying on any single conception of disease, the invention provides repeatable comparisons between samples from the individual with the reference database, where changes over time can be significant.

The invention provides methods of reporting the comparisons and their changes. One report can list the simple values of genetic information for the individual's sample at the loci for the reference specimen profile. Another report can list the instances where the values are different from the values at the loci of the profile, and this can be presented in the form of a heatmap of differences at selected loci or across individual chromosomes, for example. In other embodiments, the extent of differences is reported, as determined from a region or from a set of profiles, whether related by tissue type, organ system, or not. For example, similarity or difference can be reported in terms of percentage identity or matches or by zones determined by numerical range or by more complex formulae. The complexity of individual locus-to-locus comparisons can be deconvoluted into less complex scores to provide an overall impression or risk assessment for reporting purposes. Scores can also be provided to combine comparisons by specimen type and overall scores for portions or all of the database. The scores can represent simple averages, valuing each reference specimen profile equally, or they can be weighted to prioritize reference specimens that are suspected or have been shown to be more or less significant.

When results are available from samples taken at two or more times, the differences can be analyzed for changes in the state of the individual. Trends can be noted or tracked, and sudden changes can prompt recommendations to repeat the collection of samples or to seek professional attention for closer monitoring. When indicated, the report can include suggestions or recommendations regarding lifestyle or health to medical professionals or end-user consumers.

The information in the database can be refined as additional data become available. Certain specimen profiles can be given more or less weight when reporting comparisons, as can particular loci in a profile. The invention provides methods for providing feedback to the database based on information from the observation and treatment of the individuals. When individuals are diagnosed or treated with specific diseases or conditions, the feedback can be used to develop signature profiles within the database to provide focused comparisons for particular acquired conditions, such as metabolic disorders, metabolic syndrome, type II diabetes, and early stage cancers.

### EXAMPLES

### Example 1

### Recognition of Stage-One Lung Cancer

Plasma cfDNA was prepared and sequenced from 51 healthy individuals and from 97 individuals with stage-one cancer: bladder (7), breast (29), cervical (9), gastrointestinal (10), kidney (8), lung (26), and prostate (8). Loci from these sample sequences were used to predict their physiological state using the algorithms and lung-specific databases built using the method described in the application. The comparison recognized stage-one lung cancer samples (67% specificity), while screening out breast cancer samples (only 10% specificity). which served as negative controls. The 67% specificity can be reported as a risk assessment or further processed as a risk assessment score.

Comparisons were also performed for breast, prostate, head & neck, kidney, and blood cancers, and were able to distinguish the respective stage-one cancer types compared to the other cancer types as well as healthy controls, at varied specificity.

The headings provided above are intended only to facilitate navigation within the document and should not be used to characterize the meaning of one portion of text compared to another. Skilled artisans will appreciate that additional embodiments are within the scope of the invention. The invention is defined only by the following claims; limitations from the specification or its examples should not be imported into the claims.

I claim:

1. A method for detecting a change in the physiological state of a subject by comparison to a database of profiles from a plurality of pathological and/or nonpathological specimen samples, wherein each profile comprises reference values of a set of loci, comprising the steps of
(1) performing a comparison with a sample from a subject taken at first time point by
(a) preparing a sample of DNA from the subject;
(b) library preparation and sequencing of the DNA;
(c) comparing the sequences with the values at the set of loci in the profiles;
(2) performing the comparison of steps (a) to (c) with a sample from the subject taken at a second time point; and
(3) detecting a change if the difference between the results from step (2) differ from the results from step (1) by predetermined criteria.

2. The method of claim 1, wherein the change indicates a pathological state in the subject.

3. The method of claim 2, wherein the change indicates a pathological state that is related to a specific tissue-of-origin.

4. The method of claim 3, wherein the pathological state is selected from the group consisting of an inflammatory state, an increased risk of cancer, and an early stage cancer in the subject.

5. The method of claim 2, wherein the change indicates a pathological state in that is unrelated to a tissue of the profile.

6. The method of claim 5, wherein the change is selected from the group consisting of a tumor of a different specimen type, infection, physical injury, degeneration of the tissue, atherosclerosis, immune response of or to a tissue, metabolic change to the subject.

7. The method of claim 6, wherein the change is an autoimmune response.

8. The method of claim 1, wherein the database has profiles for at least 2 specimen types, and at least 2 profiles for each specimen type.

9. The method of claim 1, wherein a profile has more than 20,000 loci.

10. The method of claim 1, wherein at least one profile is derived from a specimen that is pathological.

11. The method of claim 1, wherein a profile is derived from a specimen that is a pathologically characterized formalin-fixed paraffin-embedded sample (FFPE)

12. The method of claim 1, wherein a profile is obtained from a tissue lysate.

13. The method of claim 1, wherein a profile is obtained by isolating nuclei from specimen cells.

14. The method of claim 1, wherein the samples are taken from the subject's blood.

15. The method of claim 14, wherein the sample is enriched for cell-free DNA.

16. The method of claim 1, wherein a change is reported in the form of a similarity value.

17. The method of claim 1, wherein a change is reported in the form of a heatmap of differences.

18. The method of claim 1, wherein a change is reported in the form of an overall risk assessment score.

19. The method of claim 1, further comprising the step of modifying the database to revise the weights of profiles.

20. The method of claim 1, further comprising the step of reducing the set of loci in a profile for comparison.
